(19) United States
(12) Patent Application Publication
(10) Pub. No.: US 2023/0023752 A1
(43) Pub. Date: Jan. 26, 2023

(54) CONTRACTILE TISSUE-BASED ANALYSIS DEVICE

(71) Applicants: Sophion Bioscience A/S, Ballerup (DK); Danmarks Tekniske Universitet, Kgs. Lyngby (DK)

(72) Inventors: Rie Kjær Christensen, Brønshøj (DK); Sandra Wilson, Birkerød (DK); Peder Skafte-Pedersen, Copenhagen S (DK); Marie Louise Lamb Busk, Hillerød (DK); Niels Bent Larsen, Rødovre (DK); Christoffer Von Halling Laier, Kgs. Lyngby (DK)

(73) Assignees: Sophion Bioscience A/S, Ballerup (DK); Danmarks Tekniske Universitet, Kgs. Lyngby (DK)

(21) Appl. No.: 17/787,437

(22) PCT Filed: Dec. 21, 2020
(86) PCT No.: PCT/EP2020/087382

§ 371 Date: Jun. 20, 2022

(30) Foreign Application Priority Data
Dec. 20, 2019 (DK) PA 2019 70804

(51) Int. Cl. G01N 33/50; B01L 3/00; G06T 7/00

(52) U.S. Cl. CPC G01N 33/5082; B01L 3/508; G06T 7/0012; B01L 2300/0654; B01L 2300/0848; B01L 2200/16; G06T 2207/30072; G06T 2207/30048; G06T 2207/30024; G06T 2207/10016

(57) ABSTRACT

A contractile tissue-based analysis device is provided, in which a strip of contractile tissue is supported by support structure. The support structure comprises a substantially planar base element, and first and second support pillars extending from said base element. An optical detection device is arranged on the side of the base element opposite to said support pillars, and is arranged to capture image data from at least one of the head portions of the support pillars. The motion of the support pillars induced by the strip of contractile tissue can thus be captured from below, i.e. through the planar base element.

### TECHNICAL FIELD

The present technology relates to a contractile tissue-based analysis device, in which a strip of natural or engineered contractile tissue is supported by support structure. The support structure comprises a substantially planar base element, and first and second support pillars. The motion of one or both support pillars induced by the strip of contractile tissue can be captured from below, i.e. through the planar base element.

### BACKGROUND

Contractile tissues such as engineered muscle tissue strips (MTS) are of broad interest in application areas such as drug screening, individualized medicine, disease modeling, and tissue grafts. In particular, drug candidates in a drug development pipeline must be screened for adverse cardiac effects. This is most conveniently performed in vitro on engineered cardiac muscle tissue.

One of the most important and best understood functions of cardiac muscle tissue is its contractile properties. A number of studies have described the in-vitro growth of 3D, strip-format, force-generating engineered heart tissues (EHT). Analysis of the contractile properties of in-vitro grown cardiac muscle tissue enables testing of changes in human heart function without human testing. Among other things, such tests can be used to provide a quick, early indication of the potential of a drug to cause cardiac-related side-effects such as drug-induced atrial fibrillation.

Compliant materials made from biological or synthetic macromolecules are widely applied in the life science field, including in analytics and advanced cell culture. For instance, hydrogels offer tunable protein and cell adhesion properties, widely variable mechanical properties, and controllable diffusivity of dissolved compounds. Compliant materials such as hydrogels have traditionally been cast into their targeted final 3D shape, which limits the attainable design freedom. The recent emergence of 3D printing methods enables direct and fast manufacture of highly complex 3D shapes. Major 3D printing methods for compliant materials include mechanical extrusion of polymer solutions (bioprinting) and spatially selective photochemical cross-linking (stereolithography) of macromolecules.

A main challenge in contractile microtissue engineering is the robustness of the constructed tissues against ‘necking’ behavior leading to subsequent failure. Previous studies have shown the importance of the pillar stiffness and matrix composition on the robustness of the engineered tissue. However, the effects of geometrical features of the pillar itself have not been extensively studied, likely due to the limited 3D design freedom of conventional molding approaches, such as those of Hansen et al. Circulation Research Jul. 9, 2010 35-44.

Other publications on similar technologies include US2015/0313704 and Lari et al., Experimental Eye Research 94 (2012) 128-135

At least some of the above problems are addressed with the present technology.

### SUMMARY

The present invention provides a contractile tissue-based analysis device, said device comprising at least one support structure; said support structure comprising:
a substantially planar base element;
first and second support pillars, each support pillar extending from said base element in a direction substantially perpendicular to the plane of said base element;

wherein each support pillar comprises a stem portion and a head portion, in which each stem portion extends between the base element and each of said head portions;

wherein at least one, and preferably both, of said support pillars can flex along an axis Y-Y extending between the head portions of said pillars;

wherein a strip of contractile tissue extends between the head portions of said first and second support pillars;

said analysis device further comprising an optical detection device arranged on the side of the base element opposite to said support pillars,

wherein the head portion of at least one support pillar, and preferably both support pillars, comprises at least one fiducial marker which can be detected by said optical detection device;

said least one fiducial marker having an extension from the head portion at least in a direction substantially parallel to the plane of said base element,

wherein said least one fiducial marker being optically discernible by said optical detection device from the side of the base element opposite to said support pillars; and,

said optical detection device being arranged to capture image data from at least one of the said least one fiducial markers of said head portions.

Also provided is a method for analysing the response of a strip of contractile tissue to a drug, said method comprising the steps of:
providing an analysis device according to the invention;
capturing a first set of image data from at least one fiducial marker of at least one of the head portions of said first and second support pillars by means of said optical detection device,
introducing said drug to said analysis device such that it contacts the strip of contractile tissue;
capturing a second set of image data from at least one of the head portions of said first and second support pillars by means of said optical detection device.

Additional aspects of the invention are provided in the dependent claims, the figures and the following description text.

### LEGENDS TO THE FIGURES

FIG. 1A shows an embodiment of a support structure, without contractile tissue.

FIG. 1B shows another embodiment of a support structure, without contractile tissue.

FIG. 1C shows another embodiment of a support structure, without contractile tissue.

FIG. 1D shows an embodiment of a support structure, without contractile tissue, and viewed along axis Y-Y extending between the head portions of the pillars.

FIG. 2 shows an analysis device based on the support structure of FIG. 1A

FIG. 3 shows support structure designs containing wells with integrated pillars for tissue formation. (a) Micro-well support structure with 170 micro wells using rectangular pillars. (b) Mini-well support structure with 10 mini-wells using rectangular pillars. (c) Support structures with 10 mini-wells using teardrop-shaped pillar heads which are engineered to minimize local stress in the forming muscle microtissue. The design additionally includes 3D triangular fiducial markers at the head to facilitate automated optical tracking of the pillar end displacement. Scale bars 2 mm (top row) and 500 μm (bottom row).

FIG. 4 shows tissue formation is observed over 4 days from cell loading into the support structure designs of FIG. 3 (a), (b) and (c), until compacted tissues are observed at day 3. White scale bars 250 μm. Black scale bars 500 μm.

FIG. 5a shows an alternative design of the analysis device to that of FIG. 3c, in top view. Three identical support structures are arranged in a “three-leaf clover” arrangement (only one of which is shown in full). Each support structure comprises a pair (first and second) of support pillars, the centrally-arranged one of which can flex along an axis Y-Y extending between the head portions 22, 32 of said support pillars. Each head portion 22, 32 in FIG. 5a includes two fiducial markers 26, 36 protruding at an angle of approximately 45 degrees from the vertical. A stationary fidicual marker is defined as a reference point (the central cross-shape), where the three support structures merge. FIG. 5 also shows planar base element 10 and receptacle 40.

FIG. 5b shows the bottom view (inverted microscope configuration) of the 3D printed muscle tissue analysis device of FIG. 5a. A muscle tissue consisting of C2C12 mouse myoblasts has formed between one pair of support pillars. Both the sloped moving fiducial markers and the horizontal stationary fiducial markers are optically distinct features in the inverted microscope image, even after introduction of the muscle tissue.

FIG. 6 shows the fidicual markers on the heads being automatically identified by the tracking software and marker by crosses at three time points (a, b and c) during muscle tissue contraction. Scale bar 500 μm

FIG. 7a shows an alternative design of the analysis device to that of FIG. 3c and FIG. 5a, in top view. A support structure comprising a pair (first and second) of support pillars, each of which can flex along an axis Y-Y extending between the head portions of said support pillars. Each head portion in 7a includes two fidicual markers protruding at an angle of approximately 45 degrees from the vertical, as well as an embossed fidicual marker on the head. FIG. 7b shows the bottom view of the 3D printed analysis device of FIG. 7a, with a muscle tissue consisting of C2C12 mouse myoblasts formed between the support pillars 3 days after culture. The focal plane of the microscope objective is adjusted to coincide with endpoints of the protruding fiducial markers. The protruding fiducial markers are clearly outlined. FIG. 7c shows the same device at the same timepoint as in FIG. 7b, but with the focal plane adjusted to coincide with the plane of the embossed markers on the support pillars. The embossed markers are barely detectable. FIG. 7d shows the same device after 4 days of culture with further compaction of the muscle tissue and stronger associated deflection of the support pillars. The focal plane is adjusted to coincide with the endpoints of the protruding fiducial markers that remains clearly outlined. The embossed fiducial markers on the support pillars cannot be discerned for this extent of tissue compaction and pillar deflection. Scale bar 500 μm.

### DETAILED DISCLOSURE

A contractile tissue-based analysis device is thus provided, which may be used to rapidly analyse the contractile properties of contractile tissue; in particular with regard to its response to drugs.

The invention allows automated analysis of the contractile behaviour of contractile tissues such as muscle tissues. This is relevant for performing in vitro screening of toxicity and effect of candidate medication industrially and in clinical settings. The invention involves a design and a fabrication method to enable optical tracking of the contraction behaviour in an inverted microscope configuration. This is industrially an important improvement over prior art describing how to track the contraction in an upright microscope configuration, since it allows for contraction monitoring without compromising tissue sterility and does not obstruct access to the tissue under study.

The focus of this technology is on the “inverted geometry”, in that support pillar deflection is observed from the side of the support structure where they are anchored (i.e. below). The results from Hansen et al. (Circulation Research, Jul. 9, 2010, pages 35-44) quite clearly show the challenges in tracking the post positions in that configuration; thus, Hansen et al. need to track on the visible part of the muscle strip itself which is clearly not optimal in an automated industrial setting.

### Support Structure

The analysis device 200 comprises at least one support structure 100. The support structure is that component of the device which supports contractile tissue, and the deflection of which can be detected by optical means. The support structure allows in-situ growth and support of contractile tissues such as cardiac muscle tissue, while allowing said contractile tissue to contract autonomously or in response to a stimulus.

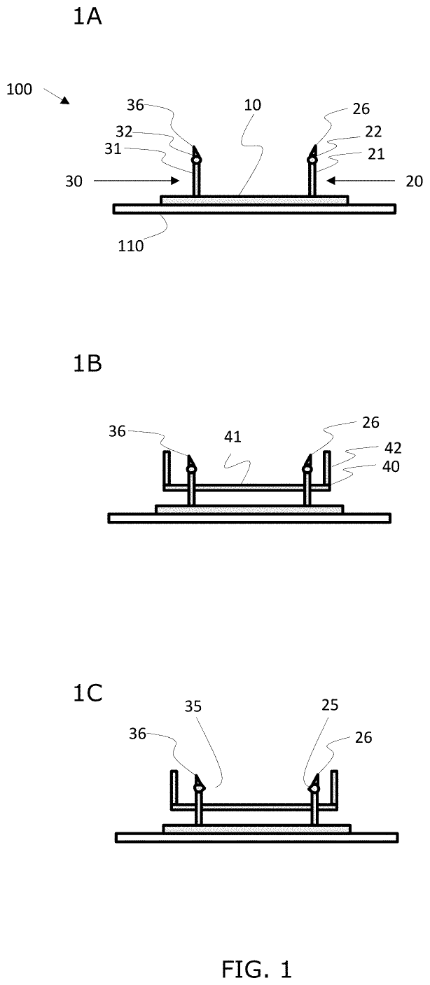

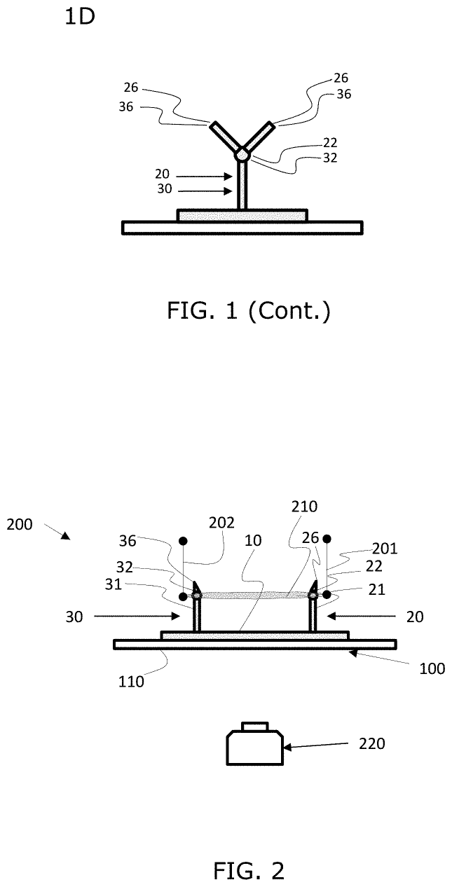

Embodiments of the support structure are illustrated in FIG. 1A-1C. In general terms, the support structure comprises: a substantially planar base element 10; and first 20 and second 30 support pillars. Additional support pillars may be present as required, and—in one particular embodiment—the support structure may additionally comprise 1 or more, such as 2, 3 or 4 additional support pillars.

The substantially planar base element of the support structure provides a base upon which the support pillars are arranged. In that the base element is “substantially planar”, it extends between substantially parallel upper and lower surfaces, and the extension between upper and lower surfaces is significantly smaller than the extension of these surfaces. The upper and lower surfaces of the base element may be e.g. rectangular, square, circular or oval shaped, or any other suitable shape. Typically, the base element has a thickness of 0.1-1.0 mm. Typically, the base element has a maximum extension in its plane of 2-40 mm. The base element is suitably formed of a polymer matrix; preferably the same polymer matrix as the support pillars.

The support pillars 20, 30 of the support structure extend from the base element 10 in a direction substantially perpendicular to the plane of said base element. This direction is generally defined as being “above” the plane of the base element. Each support pillar comprises a stem portion 21, 31 and a head portion 22, 32, in which each stem portion 21, 31 extends between the base element 10 and each of said head portions 22,32. Preferably, the head portion of each support pillar has a distinct form, which differentiates it from the stem portion, although head portion and stem portion may have the same form. Importantly, at least one, and preferably both, of said first and second support pillars can flex along an axis Y-Y extending between the head portions of said pillars. Due to the flexibility of the support pillars, any contraction of contractile tissue, autonomously or in response to a stimulus, is transferred to the head portions of the support pillars, and this can—in turn—be detected by the analysis device. Suitably, the axis Y-Y is arranged substantially parallel to the plane of said base element; making optical tracking of the head portions and subsequent analysis easier.

The stiffness of the support pillars, determined as the inverse of the pillar flexibility (compliance), is preferably in the range of 0.01-10 N/m, for example in the range 0.1-1 N/m.

Suitably, the head portions of the first and second support pillars are separated from each other along the axis Y-Y a distance less than or equal to 3 mm, e.g. less than 2 mm, or less than 1 mm. This arrangement allows improved viability of contractile tissue cultured between the head portions, since the path length for the essential continued diffusion of oxygen to all parts of the contractile tissue will be shorter for tissue of smaller dimensions.

First and second support pillars typically have the same three-dimensional form. In the embodiment shown in FIG. 1A, the head portions are substantially spherical, and the stem portions are substantially cylindrical. Suitably, in this embodiment, the axis of each cylindrical stem portions extends through the geometric centers of each spherical body of the head portions.

In the preferred embodiment shown in FIG. 1C, each head portion has a three-dimensional teardrop shape, with a substantially spherical body which extends to a vertex 25, 35. The head portions of each support pillar are arranged such that the vertexes of each head portion and the geometric centers of each spherical body, are located along the same axis Y-Y extending between said head portions. The vertexes of each head portion are located inward of said geometric centers of each spherical body along said axis Y-Y.

Teardrop-shaped pillar heads according FIG. 1C provide an advantage over rectangular pillars by not introducing the same degree of stress concentrations around sharp corners. This reduces the risk of the thinning and ultimately breaking of the tissue that is seen in the tissues of FIGS. 4a and 4b. Muscle tissue strips (MTS) created in the support structures according to the invention using teardrop-shaped heads with soft-edged biomechanical cues show no tissue damage at day 3 of culture. This MTS also exhibit a more defined tissue formation as seen in FIG. 4c.

The support pillars are suitably formed of a polymer matrix; preferably the same polymer matrix as the base element.

The analysis device of the invention includes stationary or moving optical markers—fiducial markers—introduced during fabrication of the two force responsive pillars and between which the muscle tissue is formed and held. The fiducial markers can be three-dimensional objects defined either by the selective omission of material (embossed structures) or selective addition of material (protruding structures) in 3D. The fiducial markers can be defined as having a major axis being perpendicular to the plane of said base element, or be defined with its major axis or major axes being neither parallel nor perpendicular to the plane of said base element, such as the two rectangular prisms protruding at opposing angles from the support pillars.

Therefore, the support structures may comprise fiducial markers 26, 36 for optical tracking. The incorporation of fiducial markers enables accurate contraction analysis of the muscle tissue. When tissues form around the pillar at a defined position with respect to the head portion, the height of tissue attachment is also well-defined. This enables reliable calculation of the contractile force based on pillar stiffness and deflection. The length of deflection can be determined by optical tracking of the incorporated fiducial markers, thus enabling calculation of the force exerted by the cells in the engineered muscle tissue strip.

High-resolution 3D printing enables the introduction of such non-vertically extruded fiducial markers of sub-millimeter dimensions during device fabrication. The ability to manufacture such fidicual markers by 3D printing at relevant length scales and in sufficiently compliant materials constitutes part of the present invention. This is a further advantage over the prior art of Hansen et al. (ibid) in which pillars are moulded.

Therefore, the head portion 22, 32, of at least one, and preferably each, support pillar 20, 30 suitably comprises at least one fiducial marker 26, 36 which can be detected by the optical detection device. To improve optical tracking of the head portions, each head portion may comprise two or more fiducial markers. If two or more fiducial markers are present on one head portion, these preferably extend in different directions from the head portion. In one aspect, the head portion of each support pillar suitably comprises at least one fiducial marker. In another aspect, the head portion of one support pillar comprises at least one fiducial marker, the movement of which is tracked against another, stationary fiducial marker which is present elsewhere on the support structure or analysis device.

Optical tracking of the fiducial markers from “below” the plane of the base element is possible when the fiducial markers extend from the head portion, so that they are visible from below the plane of the base element. Visibility of fiducial markers and the head portion from below the plane may be further hindered by the presence of a contractile tissue enclosing parts of the head portion. Therefore, at least one fiducial marker 26, 36 has an extension from the (respective) head portion 22, 32 (upon which it is arranged) at least in a direction substantially parallel to the plane of said base element 10, and, preferably, also in a direction substantially perpendicular to said axis Y-Y.

In a particular embodiment, shown in FIG. 1D, the fiducial markers and the head portion of each support pillar form a “Y”-shape, when viewed along the axis Y-Y extending between the two head portions. Preferably, fiducial markers, head portion and stem portion of each support pillar are essentially co-planar, in a plane extending perpendicular to the plane of the base element. This design provides good visibility of the fiducial markers from “below” the plane of the base element. At the same time, if the markers and the pillar for a Y-shape, and the initial focus is on the lower parts of the markers, a higher part of the markers will come into sharp focus when the pillar is bent. Thus, a sharp focus can be maintained for a wide range of contractions in a Y-shaped configuration.

In the analysis device according to this embodiment, the least one fiducial marker may have a primary extension from the head portion which is aligned at an angle of between 30-60°, preferably between 40-50°, more preferably 45° to an axis which extends substantially perpendicular to the plane of the base element along each support pillar. In particular, at least one fiducial marker suitably extends from the head portion in a direction away from the base element.

The support structure of the analysis device may comprise a translucent slide 110 arranged on the face of the base element opposite to said support pillars (i.e. “below” the support structure). The optical detection device 220 is arranged on the side of the translucent slide opposite to said substantially planar base element (i.e. below the translucent slide). The translucent slide serves as additional support for the support structure. The support structure can be formed on the translucent slide, and then the support structure including the translucent slide can be incorporated in the analysis device. Suitably, at least the planar base element and the translucent slide (when present) are at least partly transparent to visible light. Preferably, the support pillars and the fiducial markers, are also at least partly transparent to visible light. In the present context, “visible light” means electromagnetic radiation having a wavelength between 400 nm and 700 nm.

As illustrated in FIG. 1B and 1C, the support element may further comprise a receptacle 40. The receptacle comprises a basewall 41 and at least one sidewall 42. The basewall comprises at least two openings, and the stem portion of each support pillar extends through one of said openings. The at least one sidewall extends from the basewall in a direction away from the base element such that receptacle defines an inner volume, wherein the head portions are fully located within said inner volume. The receptacle supports stem cells, contractile progenitor cells, or contractile tissue cells as well as relevant stromal cells in their growth medium and gel forming components prior to tissue formation.

The support structures may comprise microstructures which promote tissue formation. In one aspect, microstructures can induce directionality in the tissue forming from the along the axis between the pillars. In a second aspect, microstructures can induce tissue formation at a predetermined position on the support pillars by the presence of a localized reduction or expansion of the pillar cross-section to limit sliding of the tissue. In a third aspect, microstructures can reduce excessive tensile stress in the tissue forming by providing support of gradually changing dimensions along the axis between the pillars, for example in the format of teardrop-shaped heads with their long axis parallel to the axis between the pillars.

The support structures can be manufactured using 3D printing. 3D printing allows for a broader design spectrum and gives the possibility to further explore the mechanically induced tissue differentiation possibilities. Therefore, in one aspect, the base element, the support pillars, fiducial markers and the receptacle are formed, preferably 3-D printed, from the same polymer matrix. The polymer matrix is preferably a compliant polymer matrix, more preferably a hydrogel polymer matrix e.g. poly(ethylene glycol)-based polymer matrix.

Contractile tissue

A strip of contractile tissue 210 extends between the head portions 22, 32 of said first and second support pillars 20, 30. The contractile tissue is muscle tissue, such as e.g. cardiac muscle tissue, skeletal muscle tissue or smooth muscle tissue. Of these, cardiac muscle tissue is preferred.

Muscle tissue can be formed on the support structure described above using the following method steps:
seeding the support structure described herein with muscle tissue progenitor cells or muscle tissue cells, in a culture medium comprising components to support cell survival and components to induce medium gelation, being either a mixture of fibrinogen, aprotinin, and thrombin, or a mixture of gelatin methacryloyl and a free radical photoinitator, or a mixture of collagen type I and bicarbonate;
incubating or illuminating said support structure and muscle tissue cells until a tissue is formed;
culture said muscle tissue cells, suitably for a period of 2-40 days, and stimulating the formed tissue via a combination of one or more of electrical, metabolic and mechanical stimulation,
thus providing a support structure as described herein having a strip of muscle tissue extending between the head portions of said first and second support pillars.

The cells used in the present technology are suitably mammalian stem cells, progenitor cells, or muscle tissue cells, and preferably human stem cells, progenitor cells, or muscle tissue cells. When the cells are derived from stem cells, the stem cells may be of foetal or non-foetal origin, preferably non-foetal, such as dermal skin cells (fibroblasts). The cells used in the following examples are either human induced Pluripotent Stem Cell-derived cardiac progenitor cells (hiPSC-cardiomyocyotes) or mouse myoblast cells (C2C12).

### Optical Detection Device

The analysis device 200 further comprises an optical detection device 220 arranged on the side of the base element opposite to said support pillars (i.e. below the support structure). The analysis device is arranged to capture image data from at least one of the head portions of said first and second support pillars. Suitably, the optical detection device is a camera, such as a video camera, preferably a CMOS or CCD based image sensor with a 1× objective. The image data is preferably video image data. The image data can be processed using computer software to analyse contraction of the muscle tissue.

The placement of the optical detection device below the support structure provides the advantage that the muscle tissue can be grown in-situ in the device, and then analysed directly, without disturbing the tissue and without moving the support structure. Additionally, optical detection and associated electronics can be arranged remote from the “wet” upper side of the support structure (where cell growth media and drugs are introduced to the support structure).

### Analysis Device

A schematic illustration of the analysis device is shown in FIG. 2, based on the support device of FIG. 1A.

The analysis device may further comprise a pair of electrodes connected to a power supply and arranged to apply electrical stimulation to the strip of cardiac muscle tissue. These are used to induce contractions in the muscle tissue, when measuring a response to a drug. Additionally, electrical stimulation can be used when maturing muscle tissue from stem cells.

The analysis device may further comprise at least one dosing means for providing at least one drug to said strip of muscle tissue.

### Array

An array, e.g. a 96-well platform is provided for the fabrication, culture and analysis of muscle tissue.

The analysis device described herein, may therefore comprise a plurality of support structures arranged in a planar array; in which the planar base elements of all support structures in the array are substantially co-planar in the plane of said array; and, wherein the support pillars of all support structures in the array are arranged on the same face of the planar array. Such an array provides the potential for rapid-screening of the response of muscle tissue to drugs.

In one aspect, the analysis device is a multiwell plate, said multiwell plate having substantially planar upper and lower faces and comprising a plurality of wells being open at said upper face; wherein at least one well—and preferably all wells—of said multiwell plate comprise the support structure as defined herein.

In this array, a single optical detection device may be arranged on the side of the planar array opposite to the support pillars, which optical detection device is moveable in the plane of said array. In this manner, a single optical detection device can be used to analyse multiple strips of muscle tissue.

As an alternative, in this array, a plurality of optical detection devices may be arranged on the side of the planar array opposite to the support pillar, wherein each optical detection device is arranged to capture image data from at least one of the head portions of said first and second support pillars of each support structure.

A method is also provided for analysing the response of a strip of contractile tissue to a drug, said method comprising the steps of:
providing an analysis device as described herein;
capturing a first set of image data from at least one fiducial marker of at least one of the head portions of said first and second support pillars by means of said optical detection device,
introducing said drug to said analysis device such that it contacts the strip of cardiac muscle tissue;
capturing a second set of image data from at least one of the head portions of said first and second support pillars by means of said optical detection device.

Advantageously, the strip of contractile tissue is paced by application of a pulsed electrical signal at least during capture of said first and said second set of image data.

The “response” analysed above may be the contractility response of a strip of contractile tissue and the second set of image data may be characteristic of the contractility response of said strip of contractile tissue to said drug. The first and/or said second set of image data may be video image data.

### EXAMPLES

### Materials and Methods

### Stereolithographic 3D Printing and Printing Solution Composition.

3D support structures made from poly(ethylene glycol)diacrylate Mn 700 g·mol⁻¹ (PEGDA, 455008, Sigma-Aldrich) hydrogels are obtained by projection stereolithography using a high resolution 3D printer. The printer uses one-to-one projection of dynamic images displayed on a Digital Mirror Structure (DMD) with a pixel pitch of 10.8 μm in both lateral dimensions. The aqueous printing solutions contain 5 mg/mL photoinitiator (lithium phenyl-2,4,6-trimethylbenzoyl phosphinate, LAP, Allevi or 900889, Sigma-Aldrich) and 9 mg/mL photoabsorber (quinoline yellow, QY, 309052, Sigma-Aldrich) dissolved in ultrapure MilliQ water (MQ, Merck-Millipore) with either 200 mg/mL PEGDA (‘20% PEGDA’) or 500 mg/mL PEGDA (‘50% PEGDA’). The solution components are mixed at room temperature and degassed for 30 min to avoid bubbles that would interfere with the initiator light and cause deformations in the 3D printed object.

Computer aided design (CAD) structures are drawn using Autodesk Inventor Professional with dimensions fitting a multiple of the pixel pitch (10.8 μm) of the printer's DMD. This secures the best possible dimension accuracy of the printed subject compared to the CAD design dimensions. The CAD structure is sliced with a thickness of 20 μm using the open source Slic3r software (www.slic3r.org). The sliced structure is 3D printed with 365 nm light with an intensity of 20 mW/cm2 using 3 s or 5 s of light exposure for 50% PEGDA and 20% PEGDA, respectively. The structure is 3D printed on a surface treated glass cover slip (22×22 mm #4, Menzel-Gläser). The surface treatment provides a methacrylate layer on the cover slip to enable chemical crosslinking between the print and the glass cover slide.

### Preparation of Support Structures.

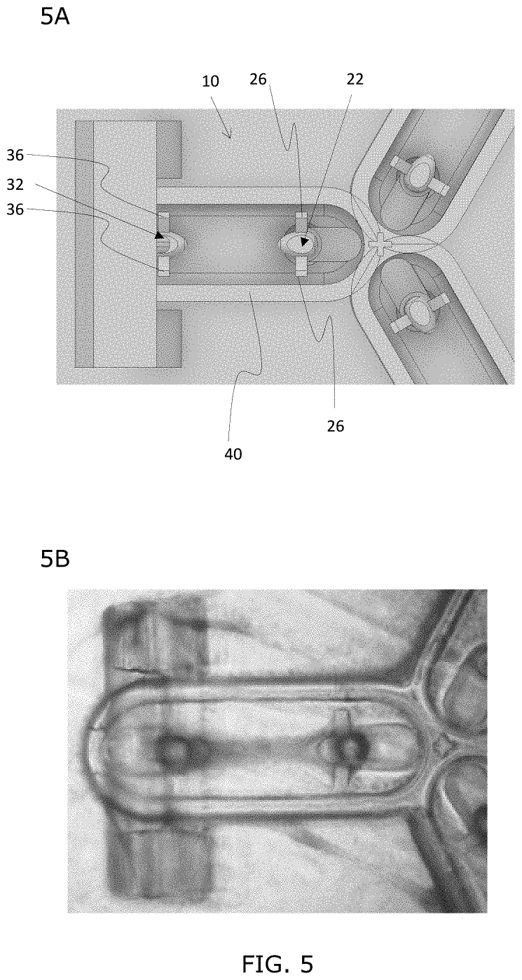

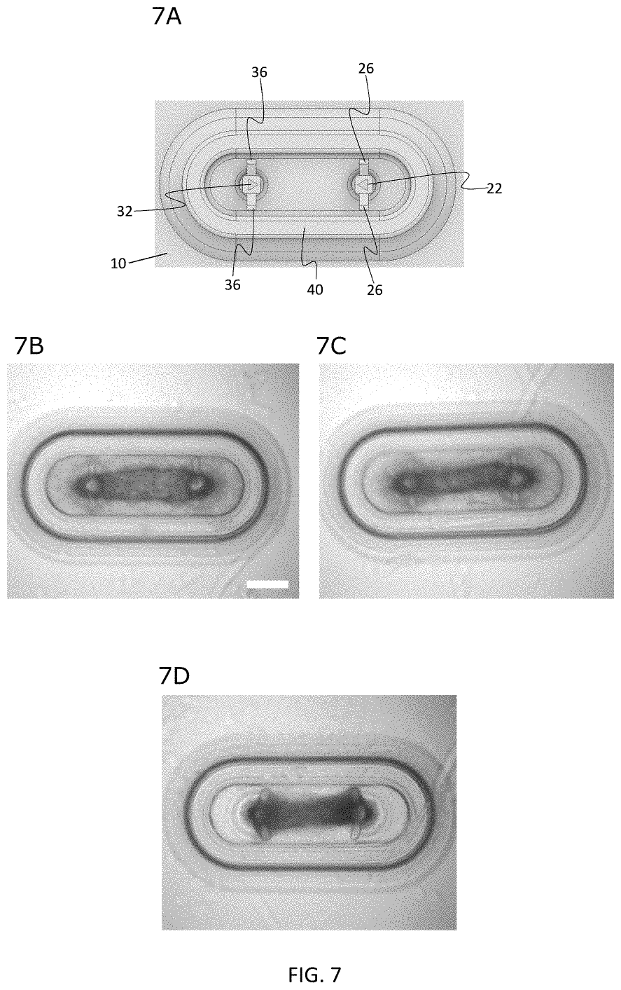

Five different support structures are produced for generating Muscle Tissue Strips (MTS). Two designs are selected from previously investigated cell seeding platforms with the use of support pillars (FIGS. 1a and b). The third design (FIG. 1c; according to the invention) is developed to minimize stress concentrations around the vertical support pillars to promote a more robust MTS formation. The fourth design (FIG. 5a; according to the invention) includes fiducial markers extending at opposing angles from the head of the pillars as well as a stationary reference marker. The fifth design (FIG. 7a, according to the invention) presents fiducial markers extending from the pillar heads, as in the fourth design, and additionally embossed fiducial markers on top of the pillar heads. 3D printed support structures are washed in phosphate buffered saline (PBS) for at least 24 h after printing. The liquid is exchanged two times to wash out any residual print solution from the cross-linked PEGDA network. Sterilization is performed by immersing the printed support structures in 70% v/v ethanol/water for 10 minutes followed by UV-C exposure (254 nm) for 15 minutes (Mini UV Sterilisation Cabinet, Cleaver Scientific). The support structures are stored sterile in PBS until use to ensure exchange of water to PBS prior to cell culture. Before seeding cells into the support structures, the PBS is removed and the platforms are blotted dry with sterile lint free paper to make sure the wells are empty.

### Cell Seeding and Culture.

C2C12 mouse myoblasts (C3H muscle myoblast, 91031101, Sigma-Aldrich) are used. Cells are kept in culture using growth medium composed of DMEM high glucose (Sigma-Aldrich) with 10% fetal bovine serum (FBS) (Sigma-Aldrich), and 1% Penicillin/Streptomyocin (P/S, Sigma-Aldrich). Tissue formation is initiated by casting cells suspended at 10×10⁶ cells/mL in a solution of 10 mg/mL fibrinogen (F8630, Sigma-Aldrich), 0.5 μg/mL aprotinin (A1153, Sigma-Aldrich), 20% (v/v) Matrigel (354277, Corning), and 3 U/mL thrombin (T7513, Sigma-Aldrich) in growth medium into the prepared support structures. The solution is kept on ice to prevent gelation until casting. The larger support structures wells (FIG. 1b and FIG. 1c) are filled individually with 3.5 μL of the cell suspension. The smaller support structures wells (FIG. 1a) are filled in a two-step process. First, 200 μL of cell suspension is loaded on top of all wells of the support structures. Second, the support structures is spun in a centrifuge at 200 g for 10 s to force the suspension into the wells. The loaded support structures are incubated at 37° C. for 30 min to let the fibrin matrix form before growth medium is added. After 2 days in culture, the medium is changed to DMEM high glucose with 2% FBS and 1% P/S to enhance the fusion of myoblasts to myotubes. Medium change is conducted every 2-3 days throughout the culture time.

### Viability Staining.

Staining is performed by incubating for 1 h at 37° C. with 2 μg/μL Calcein AM (15560597, Fisher Scientific), 4 μg/mL propidium iodide (81845, Sigma-Aldrich), and 2 μg/mL Hoechst 34580 (H21486, Invitrogen). Samples are then washed with medium before confocal imaging on a Zeiss LSM700 using a Zeiss 10×/0.3 NA Epiplan Neofluar objective with excitation at 405, 488, and 555 nm for Hoechst 34580, calcein AM, and propidium iodide, respectively. The recorded z-stacks are collapsed to a 2D image in FIJI/ImageJ by maximum intensity projection.

### Formation of Muscle Tissue Strips (MTS).

Mouse myoblasts are cast at 10×10⁶ cells/mL in a fibrin/Matrigel matrix in the three different support structures. The cells will start to differentiate when they are in close proximity to each other. Upon differentiation, the myoblasts fuse and form myotubes. Elongation of the cells is a sign of differentiation and shows alignment of the sarcomeres responsible for cell contraction. When the sarcomeres align, the cells can exert more force in the direction of the elongation. As the cell-laden fibrin matrix is embedded around the pillars, the cells' contraction of the matrix is restricted by the pillars, causing them to form an elongated structure and eventually an MTS.

The stiffness of the pillars will continue to provide an opposing force when the cells contract. After 24 h of culture, the cells have started to contract the fibrin matrix and form a tissue surrounding the two pillars (FIG. 4). Two days after seeding, well-defined tissues are seen in all designs.

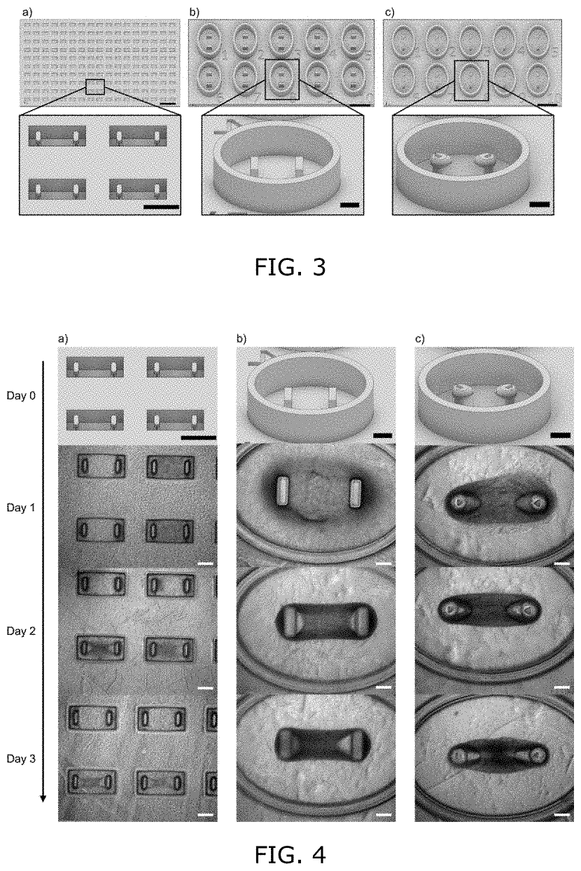

In the support structures of FIG. 3a, the formed tissue is surrounded by excess cells captured in the matrix without contributing to the tissue formation. The design of FIG. 3a requires fewer cells per well due to the small well size, but requires a highly inefficient cell seeding method with a lot of lost cells. In both the support structures of FIGS. 3a and 3b, tissues are at risk of thinning and ultimately breaking due to necking as seen in FIGS. 4a and 4b. Necking is known to cause engineered tissues to fracture regardless of cell type and poses a serious challenge in tissue engineering. Teardrop-shaped pillar heads according to the invention provide an advantage over rectangular pillars by not introducing the same degree of stress concentrations around the corners. This reduces the risk of the thinning and ultimately breaking of the tissue that is seen in the tissues of FIGS. 4a and 4b. Muscle tissue strips created in the support structures according to the invention using teardrop-shaped heads with soft-edged biomechanical cues show no tissue damage at day 3 of culture. This MTS also exhibit a more defined tissue formation as seen in FIG. 4c. All engineered tissues show a high cell viability and a tight cellular structure with aligned cells.

Support structure designs with protruding fiducial markers on the pillar heads, as shown in FIG. 5a and FIG. 7a, support MTS formation equally well as designs without protruding fiducial markers. FIG. 5b presents a micrograph in bottom view of an MTS formed in the design of FIG. 5a, while FIGS. 7b-7d displays bottom views of an MTS formed in the design of FIG. 7a at day 3 (FIGS. 7b+7c) and day 4 (FIG. 7d) of culture, respectively. FIGS. 7b through 7d illustrate how fiducial markers embossed on the pillar heads become increasingly difficult to discern with time due to optical distortion of an embossed profile by the compacting opaque tissue engulfing the pillar heads as well as the increasing deflection of the pillar heads. The profile and axial position of the protruding fiducial markers remain clearly visible at all time points. The choice of a protrusion direction of the fiducial marker not coinciding with the base plane enables high optical quality imaging of at least parts of the fiducial marker at a constant focal distance for a multitude of pillar deflections.

### Optical Analysis

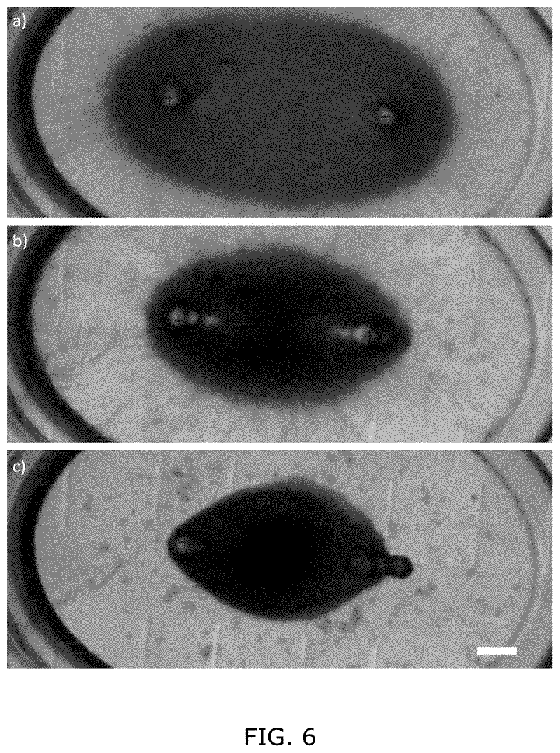

Tissue contraction of support designs in FIG. 3 is monitored using a custom-made stage incubator mounted on a Motic stereomicroscope. Images are recorded every 5 min. A custom-made tracking software is used to track the optical markers on the pillar heads in the acquired image sequences. FIG. 6 shows the fiducial markers on the heads being automatically identified by the tracking software and marker by crosses at three time points (a, b and c) during muscle tissue contraction. The scale bar is 500 micrometers.

1. A contractile tissue-based analysis device (200), said device (200) comprising at least one support structure (100); said support structure (100) comprising:
a substantially planar base element (10);
first (20) and second (30) support pillars, each support pillar (20, 30) extending from said base element (10) in a direction substantially perpendicular to the plane of said base element (10);
wherein each support pillar (20, 30) comprises a stem portion (21, 31) and a head portion (22, 32), in which each stem portion (21, 31) extends between the base element (10) and each of said head portions (22, 32);
wherein at least one, and preferably both, of said support pillars (20, 30) can flex along an axis Y-Y extending between the head portions (22, 32) of said pillars (20, 30);
wherein a strip of contractile tissue (210) extends between the head portions (22, 32) of said first (20) and second (30) support pillars;
said analysis device (200) further comprising an optical detection device (220) arranged on the side of the base element (10) opposite to said support pillars (20, 30),
wherein the head portion (22, 32) of at least one support pillar (20, 30), and preferably both support pillars (20, 30), comprises at least one fiducial marker (26, 36) which can be detected by said optical detection device (220);
said least one fiducial marker (26, 36) having an extension from the head portion (22, 32) at least in a direction substantially parallel to the plane of said base element (10),
wherein said least one fiducial marker (26, 36) being optically discernible by said optical detection device (220) from the side of the base element (10) opposite to said support pillars (20, 30); and,
said optical detection device (220) being arranged to capture image data from at least one of the said least one fiducial markers (26, 36) of said head portions (22, 32).

2. The analysis device (200) according to claim 1, wherein said least one fiducial marker (26, 36) extends beyond said strip of contractile tissue (210) and said support pillars (20, 30) at least in a direction substantially parallel to the plane of said base element (10).

3. The analysis device (200) according to any one of the preceding claims, wherein said least one fiducial marker (26, 36) also has an extension from the head portion (22, 32) at least in a direction substantially perpendicular to said axis Y-Y.

4. The analysis device (200) according to any one of the preceding claims, wherein said least one fiducial marker (26, 36) has a primary extension from the head portion (22, 32) which is aligned at an angle of between 30-60°, preferably between 40-50°, more preferably 45° to an axis which extends substantially perpendicular to the plane of the base element (10) along each support pillar (20, 30).

5. The analysis device according to any one of the preceding claims, wherein said at least one fiducial marker (26, 36) extends from said head portion (22, 32) in a direction away from the base element (10).

6. The analysis device (200) according to any one of the preceding claims, further comprising a pair of electrodes (201, 202) connected to a power supply and arranged to apply electrical stimulation to the strip of cardiac muscle tissue (210).

7. The analysis device (200) according to any one of the preceding claims, wherein the support structure (100) of said analysis device (200) comprises a translucent slide (110) arranged on the face of said substantially planar base element (10) opposite to said support pillars (20, 30) and wherein said optical detection device (220) is arranged on the side of the translucent slide (110) opposite to said substantially planar base element (10).

8. The analysis device (200) according to any one of the preceding claims, wherein said head portions (22, 32) are separated from each other along said axis Y-Y a distance less than or equal to 2 mm.

9. The analysis device (200) according to any one of the preceding claims, wherein said axis Y-Y is arranged substantially parallel to the plane of said base element (10).

10. The analysis device (200) according to any one of the preceding claims, wherein at least the planar base element (10) and the translucent slide (110), and preferably the support pillars (20, 30) and the fiducial markers (26, 36), are at least partly transparent to visible light.

11. The analysis device (200) according to any one of the preceding claims, wherein the head portion (22, 32) has a three-dimensional teardrop shape, with a substantially spherical body which extends to a vertex (25, 35) and wherein the head portions (22, 32) of each support pillar (20, 30) are arranged such that the vertexes (25, 35) of the head portions (22, 32) and the geometric centers of each spherical body, are located along said axis Y-Y, and wherein said vertexes (25, 35) of each head portion (22, 32) are located inward of said geometric centers of each spherical body along said axis Y-Y.

12. The analysis device (200) according to any one of the preceding claims, further comprising a receptacle (40), said receptacle (40) comprising a basewall (41) and at least one sidewall (42), said basewall (41) comprising at least two openings (41′), wherein each stem portion (21, 31) of the support pillars (20, 30) extends through one of said openings (41′); and wherein said at least one sidewall (42) extends from said basewall (41) in a direction away from the base element (10) such that receptacle (40) defines an inner volume, and wherein the head portions (22, 32) are fully located within said inner volume.

13. The analysis device (200) according to any one of the preceding claims, further comprising at least one dosing means for providing at least one drug to said strip of muscle tissue.

14. The analysis device (200) according to any one of the preceding claims, wherein said base element (10), said support pillars (20, 30) and said receptacle (40) are formed, preferably 3-D printed, from the same polymer matrix, preferably a compliant polymer matrix, more preferably a hydrogel polymer matrix; e.g. poly(ethylene glycol)-based polymer matrix.

15. The analysis device (200) according to any one of the preceding claims, wherein said device (200) comprises a plurality of support structures (100) arranged in a planar array (250);
wherein the planar base elements (10) of all support structures (100) in the array (250) are substantially co-planar in the plane of said array (250); and,
wherein the support pillars (20, 30) of all support structures (100) in the array are arranged on the same face of the planar array (250).

16. The analysis device (200) according to claim 15, being a multiwell plate, said multiwell plate having substantially planar upper and lower faces and comprising a plurality of wells being open at said upper face; wherein at least one well—and preferably all wells—of said multiwell plate comprise the support structure (100) as defined in any one of claims 1-13.

17. The analysis device (200) according to any one of claims 15-16, wherein a single optical detection device (220) is arranged on the side of the planar array (250) opposite to said support pillars (20, 30), said optical detection device (220) being moveable in the plane of said array (250).

18. The analysis device (200) according to any one of claims 15-17, comprising a plurality of optical detection devices (220) arranged on the side of the planar array (250) opposite to said support pillars (20, 30), wherein each optical detection device (220) is arranged to capture image data from at least one of the head portions (22, 32) of said first (20) and second (30) support pillars of each support structure (100).

19. A method for analysing the response of a strip of cardiac muscle tissue (210) to a drug, said method comprising the steps of:
providing an analysis device (200) according to any one of the preceding claims;
capturing a first set of image data from at least one fiducial marker (26, 36) of at least one of the head portions (22, 32) of said first (20) and second (30) support pillars by means of said optical detection device (220),
introducing said drug to said analysis device (200) such that it contacts the strip of contractile tissue (210);
capturing a second set of image data from at least one of the head portions (22, 32) of said first (20) and second (30) support pillars by means of said optical detection device (220).

20. The method according to claim 19, wherein said strip of contractile tissue (210) is paced by application of a pulsed electrical signal at least during capture of said first and said second set of image data.

21. The method according to any one of claims 19-20, wherein said response is the contractility response of strip of contractile tissue (210), and wherein said second set of image data is characteristic of the contractility response of said strip of contractile tissue (210) to said drug.

22. The method according to any one of claims 19-21, wherein said first and/or said second set of image data is video image data.
